(19) United States
(12) Patent Application Publication
(10) Pub. No.: US 2025/0012885 A1
(43) Pub. Date: Jan. 9, 2025

(54) A NUMERICAL METHOD FOR THE SEPARATION OF SHEAR COMPRESSION WAVES IN A DISPLACEMENT VECTOR FIELD

(71) Applicants: INSTITUT NATIONAL DE LA SANTÉ ET DE LA RECHERCHE MÉDICALE, Paris (FR); UNIVERSITE CLAUDE BERNARD LYON 1, Villeurbanne (FR); CENTRE LEON BERARD, Lyon (FR)

(72) Inventors: Rémi SOUCHON, Lyon (FR); Tristan DERUELLE, Lyon (FR); Johannes AICHELE, Pfaffhausen (CH); Stefan CATHELINE, Lyon (FR)

(21) Appl. No.: 18/708,656

(22) PCT Filed: Nov. 15, 2022
(86) PCT No.: PCT/EP2022/082049

§ 371 Date: May 9, 2024

(30) Foreign Application Priority Data
Nov. 15, 2021 (EP) 21306587.3

(51) Int. Cl. G01R 33/563; A61B 5/055

(52) U.S. Cl. CPC G01R 33/56358; A61B 5/055

(57) ABSTRACT

Nowadays, the interest to use mechanical waves in various field such medical field or geophysical field is well established. Indeed, the study of mechanical waves propagating in a medium allows usually to retrieve the properties of this medium. In solid media, a mechanical wave is composed of two components: a compression wave and a shear wave. Depending on the field of application, it may be preferable to characterize only one of the components. However, the discretization of each component of the mechanical waves may be difficult and conventional methods are not necessarily suitable for some media. The present disclosure overcomes the above drawback by proposing a new method for separating a displacement vector field U resulting from the displacement of a mechanical wave in a medium into its shear component and its compression component. Such method is particularly adapted when the components of the mechanical waves propagate with similar speed in the medium, for instance a shear wave and a slow Biot wave in a poroelastic medium.

### TECHNICAL FIELD

The present disclosure is related to the field of Mechanics. More particularly, the present disclosure is related to a method for the separation of shear and compression waves in a displacement vector field relative to a mechanical wave propagating in a medium.

### BACKGROUND ART

Nowadays, the interest to use mechanical waves in various field such medical field or geophysical field no longer needs to be proven.

Indeed, the study of mechanical waves propagating in a medium allows to retrieve the properties of this medium. For instance, in the geophysical field, the study of mechanical wave (e.g. such P-wave or S-wave) propagating in a subsoil may allow to discretize the layers of a subsoil or determine the elastic properties of the subsoil. In the medical field, the study of the mechanical wave (e.g. acoustic wave, ultrasound wave, etc.) propagating in biological tissues (of an organ for instance) may allow to retrieve the elastic properties of this biological tissues, and then determine the state of a pathology for instance. Indeed, many pathologies (e.g. hepatic fibrosis) may lead to modification of the elastic properties of biological tissues, and which may not be yet detectable at the beginning of the disease by conventional imaging methods.

When propagating in a medium (e.g. in a viscoelastic or poroelastic medium), a mechanical wave may induct the generation of two components of the mechanical wave, a compressional (or longitudinal or compression) component relative to the compression wave and a shear component relative to the shear wave.

Depending on the field of application, it may be preferable to characterize only one of the components, and vice versa. However, the discretization of each component of the mechanical waves may be difficult, and worse, the presence of an undesirable component, i.e. when only one component is wanted, may disturb the retrieving of the properties of the medium such as its elastic properties (e.g. Young or/and shear modulus).

Conventional methods to separate each component usually rely on the large difference that may exist between the speed of the compression wave and the speed of the shear wave in a medium. For instance, in soft elastic or soft viscoelastic media such as biological tissues, the compression wave travels typically 1000 times faster than the shear wave.

However, depending on the application field or the type of medium for instance, conventional methods may be not adapted anymore. For instance, it may be the case when each component of a mechanical wave (or mechanical waves) has approximately the same speed of propagation (or the same wavelength) in the medium. Such situation may happen in Shear wave Elastography or Magnetic resonance Elastography when the medium is a poroelastic medium (e.g. biological tissues of an organ). Likewise, the conventional methods may be not adapted when the acquisition systems used for acquiring each component are not adapted for acquiring such large speed difference.

Therefore, there is a need to retrieve and quantify separately each component (e.g. the compression and the shear components) of a mechanical wave propagating in a medium, which may be mandatory when seeking the elastic properties of a medium with a high precision or performing 3D image with high resolution.

### SUMMARY

This disclosure improves the situation.

It is proposed a method for separating a displacement vector field \vec{U} resulting from the displacement of a mechanical wave in a medium into its shear component and its compression component, wherein the method may comprise:
calculating a first vector field and a second vector field from said displacement vector field \vec{U} by using the Helmholtz theorem, and said first vector field being function of a potential,
and wherein the first vector field is calculated based on the potential, and wherein the second vector field is calculated from the first vector field.

Advantageously, the present solution allows to retrieve separately the two components (compression and shear components) of a mechanical wave propagating in a medium. Advantageously, even when the compression waves and the shear waves have approximately the same speed in the medium, the present solution allows to retrieve separately each component of the mechanical wave propagating in the medium. For instance, the medium may be a viscoelastic medium or a poroelastic medium or composite medium, or may be a viscoelastic soft medium or a poroelastic soft medium or composite soft medium. Advantageously, the separation of the two components enables to create compression wave elastography images, and may also allow to improve the quality of existing shear wave elastography images. Furthermore, because the compression waves (e.g. first fast compression wave and second slow compression wave-P—also called a PII wave or longitudinal wave of the secondary kind or Biot wave—in a poroelastic medium or a poroelastic soft medium) may contain information about the elastic properties of the medium, the separation and the estimation (or calculation) of each component of the mechanical wave may be helpful when determining the poroelastic properties of a medium and increase the relevance of the properties determined.

By characterizing (or characterization), it may be understood imaging or observing a mechanical wave propagating in a medium, or also it may be understood the measurement or an image of the speed of each component, for instance by generating an image of the shear wave velocity and an image of the slow Biot wave velocity.

In the present disclosure, it may be understood that a symbol accented with an arrow denotes a vector.

In the present disclosure, it may be understood that symbol ∇ denotes the nabla (or del) operator which may be understood as a vector of partial derivative operators.

In the present disclosure, it may be understood that the symbol Δ denotes the Laplace operator.

In one or several embodiments, the potential may be a vector potential or a scalar potential.

In one or several embodiments, when the potential is a vector potential, the method may further comprise:
calculating an output resulting from the application of a curl operator on a decomposition's formula \vec{U}=−∇Φ+∇×\vec{A} obtained from the Helmholtz theorem, where said ∇×\vec{A}=\vec{U_{S}} is the first vector field relative to the shear component and −∇Φ=\vec{U_{P}} is the second vector field relative to the compression component, the said output corresponding to a first Poisson Equation according to the formula ∇²\vec{A}=−∇×\vec{U},
and wherein the vector potential A may be calculated by solving numerically said first Poisson Equation.

In one or several embodiments, when the potential is a vector potential, the vector potential may be calculated by using an integral solution of Helmholtz theorem according to the formula:

$$\vec{A} ( \vec{r} , t ) = {\int }_{V} \frac{∇ \times \vec{U} ( \vec{{r}^{′}} , t )}{4 π R ( \vec{r} , \vec{{r}^{′}} )} {dV}^{′} + {∮}_{S} \frac{\vec{U} ( \vec{{r}^{′}} , t ) \times \vec{n} ( \vec{{r}^{′}} , t )}{4 π R ( \vec{r} , \vec{{r}^{′}} )} {dS}^{′}$$

wherein,
\vec{A} is the vector potential,
\vec{r} and t are the position and time, respectively, at which the vector potential \vec{A} is being calculated,
\vec{r′} is the variable of integration and represents a moving position (it moves within V in the first integral, and over S in the second integral),
R(\vec{r}, \vec{r′})=∥\vec{r}−\vec{r′}∥ is the distance between points \vec{r} and \vec{r′},
\vec{n}(\vec{r′}) is a unit vector at position \vec{r′}, normal to surface S, pointing outward from volume V,
∇×\vec{U} is the curl operator,
\vec{U}×\vec{n}=\vec{U}∧\vec{n} denotes the cross product of vectors \vec{U} and \vec{n}.

In one or several embodiments, the first vector field \vec{U_{S}} may be calculated according to the formula \vec{U_{S}}=∇×\vec{A}.

In one or several embodiments, the second vector field \vec{U_{P}} may be calculated according to the formula \vec{U_{P}}=\vec{U}−\vec{U_{S}}.

In one or several embodiments, when the potential is a scalar potential, the method may further comprise:
calculating an output resulting from the application of a divergence operator on the decomposition's formula \vec{U}=−∇Φ+∇×\vec{A} obtained from the Helmholtz theorem, where said −∇Φ=\vec{U_{P}} is the first vector field relative to the compression component and said ∇×\vec{A}=\vec{U_{S}} is the second vector field relative to the shear component, the said output corresponding to a second Poisson Equation according to the formula ∇²Φ=−∇·\vec{U},
and wherein the scalar potential P may be calculated by solving numerically said second Poisson Equation.

In one or several embodiments, when the potential is a scalar potential, the scalar potential may be calculated by using an integral solution of Helmholtz theorem according to the formula:

$$ϕ ( \vec{r} , t ) = {\int }_{V} \frac{∇ \cdot \vec{U} ( \vec{{r}^{′}} , t )}{4 π R ( \vec{r} , \vec{{r}^{′}} )} {dV}^{′} - {∮}_{S} \frac{\vec{U} ( \vec{{r}^{′}} , t ) \cdot \vec{n} ( \vec{{r}^{′}} , t )}{4 π R ( \vec{r} , \vec{{r}^{′}} )} {dS}^{′}$$

wherein,
ϕ is the scalar potential,
\vec{r} and t are the position and time, respectively, at which the vector potential ϕ is being calculated,
\vec{r′} is the variable of integration and represents a moving position (it moves within V in the first integral, and over S in the second integral),
R(\vec{r}, \vec{r′})=∥\vec{r}−\vec{r′}∥ is the distance between points \vec{r} and \vec{r′},
\vec{n}(\vec{r′}) is a unit vector at position \vec{r′}, normal to surface S, pointing outward from volume V,
∇×\vec{U} is the divergence operator,
\vec{U}·\vec{n} denotes the dot (or scalar product) product of vectors U and n.

In one or several embodiments, the first vector field \vec{U_{P}} may be calculated according to the formula \vec{U_{P}}=−∇Φ.

In one or several embodiments, the second vector field \vec{U_{S}} may be calculated according to the formula \vec{U_{S}}=\vec{U}−\vec{U_{P}}.

In one or several embodiments, solving the first Poisson Equation or the second Poisson Equation may be performed in the frequency domain.

Advantageously, solving the Poisson Equation in the frequency domain may allow to calculate \vec{U_{S}} or \vec{U_{P}} in a single operation.

In one or several embodiments, the first Poisson Equation or the second Poisson Equation may be a discrete Poisson equation.

In one or several embodiments, the medium may be any medium that allows the propagation of shear and compression waves, including a viscoelastic medium, a poroelastic medium, a poro-visco-elastic medium, a composite medium or a poro-composite medium.

In one or several embodiments, the medium may be any medium that allows the propagation of shear and compression waves, including a viscoelastic soft medium, a poroelastic soft medium, a poro-visco-elastic soft medium, a composite soft medium or a poro-composite soft medium.

In one or several embodiments, when the medium is a poroelastic medium or a poro-visco-elastic medium or a composite medium or a poro-composite medium, the displacement vector field may comprise at least one slow compression wave called Biot wave and may comprise at least one fast compression wave.

In one or several embodiments, the compression component may be relative to at least one slow compression wave called Biot wave and to at least one fast compression wave.

In one or several embodiments, wherein when \vec{U_{P}} as first vector field is determined from \vec{U_{P}}=−∇Φ and the second Poisson Equation according to the formula ∇²Φ=−∇·\vec{U}, \vec{U_{P}} may be relative to the at least one slow compression wave called Biot wave.

In one or several embodiments, the at least one fast compression wave may be determined from the at least slow compression wave.

In one or several embodiments, the medium is further a soft medium.

By soft medium, it may be understood biological tissues such tissues of organ such as liver, kidney, brain, prostate, etc.

Advantageously, the determination of at least one slow compression wave such Biot wave may allow to then determine elastic properties of the medium such stiffness, viscosity, permeability, porosity, tortuosity of the medium which may be diagnostic markers of the studied medium.

In one or several embodiments, the imaging device may be a Magnetic Resonance Imaging configured to perform Magnetic Resonance Elastography.

In another aspect, it is proposed a 3D imaging system for imaging a first vector field and second vector field comprised in a displacement field relative to a mechanical wave propagating in a medium, the 3D imaging system may comprise:
an imaging device configured to acquire the displacement vector field relative to the mechanical waves propagating in the medium,
a control system configured for acquiring at least one raw signal data comprising the displacement vector field, for separating the displacement vector field according to the present disclosure to obtain a first vector field and a second vector field, and for generating a 3D image of the first vector field and the second vector field.

In one or several embodiments, the control system may be further configured to use the 3D image of the first vector field to determine a length and/or a speed or/and an image of speed of the wave relative the first vector field, and may be configured to use the 3D image of the second vector field to determine a length and/or a speed or/and an image of speed of the wave relative the second vector field.

By image of speed, it may be understood the determination (or generation) of an image, for each vector field, where each pixel of the image represents a value of speed (or velocity) of the wave relative to the respective vector field.

In one or several embodiments, the central frequency of the mechanical wave may be comprised between 0.1 and 10⁹ Hertz.

In one or several embodiments, the mechanical wave may be a sinusoidal wave.

In another aspect, it is proposed a computer software comprising instructions to implement at least a part of a method according to the present disclosure when the software is executed by a processor.

In another aspect, it is proposed a computer-readable non-transient recording medium on which a software is registered to implement a method according to the present disclosure when the software is executed by a processor.

### BRIEF DESCRIPTION OF DRAWINGS

Other features, details and advantages will be shown in the following detailed description and on the figures, on which:

FIG. 1 illustrates schematically an example of 3D imaging system for imaging the displacement vector field of a mechanical wave propagating in a medium.

FIG. 2 describes a flow chart of the method according to the present disclosure.

FIG. 3a illustrates an image of a medium obtained by using MRE technique.

FIG. 3b show images of shear component and compression component of a mechanical wave propagating in the medium of the FIG. 3a.

FIG. 3c illustrates a shear wave velocity image obtained from images of shear component of a mechanical wave propagating in a medium such wave images of FIG. 3b.

FIG. 3d illustrates a compression wave velocity image obtained from images of compression component of a mechanical wave propagating in a medium such wave images of FIG. 3b.

FIG. 4 illustrates an exemplary architecture of a device configured for the implementation of embodiments of the proposed scheme.

### DESCRIPTION OF EMBODIMENTS

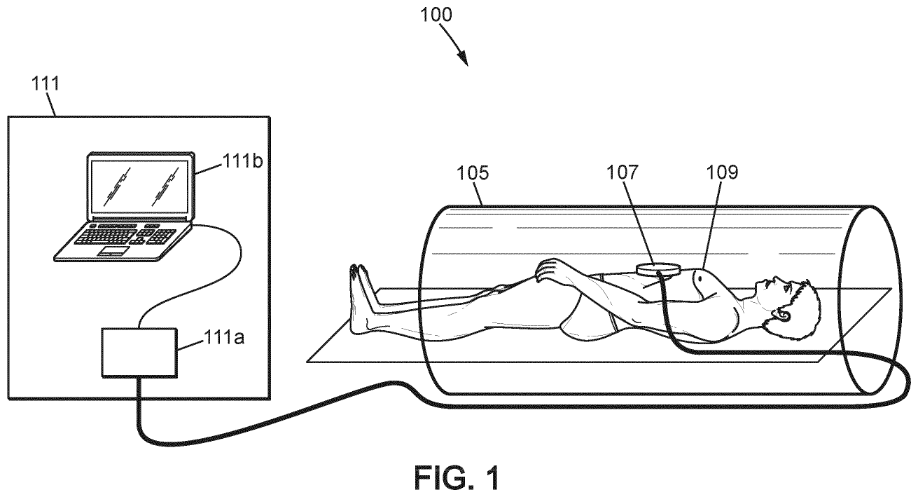

FIG. 1 illustrates schematically an example of 3D imaging system for imaging the displacement vector field of a mechanical wave propagating in a medium.

By medium, it may be understood a viscoelastic medium or a poroelastic medium, or a biological medium, or a composite medium made of several components.

The imaging system 100 shown on FIG. 1 may be configured to perform 3D (or 4D) imaging of a region of a medium. The viscoelastic medium or poroelastic medium may be the biological tissues of an organ or part of organ of a living being (e.g. human patient), such a liver or a heart or a kidney or a brain.

The 3D imaging system may be configured to acquire a displacement field of a mechanical wave propagating in a medium.

According to an example, the 3D imaging system may be configured to perform Magnetic resonance elastography (MRE).

In this purpose, the 3D imaging system 100 may comprise a Magnetic Resonance imaging (MRI) Scanner 105 configured to image the displacement fields of mechanical waves propagating in a medium according to the MRE method. The use of MRI scanner allows to record the three spatial components and the time component of wave field displacements in each voxel of a 3D volume. The full 3D (in space) displacement data may be retrieved over time.

The 3D imaging system may also comprise a mechanical wave generator 107 configured to generate and transmit one or a plurality of mechanical waves in a medium. According to an example, the mechanical wave generator may be a drum-like vibrator, or a probe comprising a plurality of transducers (e.g. piezoelectric transducers) positioned at the surface of the medium, for instance the chest of a human body 109. The medium to image may be the liver or the kidney of the patient for instance.

In one or several embodiments, rather than using a mechanical wave generator, the 3D imaging system may be configured to image the displacement fields of mechanical waves propagating in a medium which are generated by the breathing, or/and the heartbeat, or/and the voice, or/and the muscle movements, or/and any internal or/and external vibration which can be detected by the 3D imaging system.

The frequency of the mechanical waves (or central frequency of the mechanical waves) may be comprised between 0.1 and 109 Hertz for instance.

In one or several embodiments the frequency of the mechanical waves (or central frequency of the mechanical waves) may be comprised for instance in the audible range, between 20 Hz and 20 kHz, or in the ultrasound range, between 20 kHz and 1 GHz, or in the infrasound range, between 0.1 and 20 Hz for instance.

In one or several embodiments, the mechanical waves may be sinusoidal waves or pulsed waves. When using sinusoidal or pulsed vibrations, the full 3D (in space) displacement data may be retrieved over time.

Furthermore, the 3D imaging system 100 may comprise a control system 111 which may be programmed (or configured) such that the mechanical waves (or pulsed waves) are synchronized with the imaging system, for example a Magnetic Resonance Imaging (MRI) scanner, and the mechanical waves are transmitted at a rate that matches the repetition time of the imaging system, or a multiple of said repetition time. For example, the repetition rate can be between 10 milliseconds and 10 seconds.

The control system 111 may, for instance, include a control unit 111a and a computer 111b. In this example, the control unit 111a may be used for controlling the drum-like vibrator and acquiring a raw signal data from the Magnetic Resonance imaging (MRI) Scanner 105, the raw signal data comprising information data relative the displacement vector field of the mechanical wave (or mechanical waves) propagating in the medium of the human body 109.

The computer 111b may be used for controlling the control unit 111a, for processing the raw signal data acquired by the control unit 111a according to the wave separation method of the present disclosure, and for generating 3D or 2D images or movies from the filtered raw signal data. The 3D generated images may be images relative to one or a plurality of shear components and/or one or a plurality of compression components of the mechanical wave propagating in the medium. Furthermore, quantifications parameters such elastic properties (e.g. Young and/or shear modulus) of the medium (e.g. biological tissues) may be determined from the generated 3D images and by using any known inversion algorithms for instance. For example, in an elastic or viscoelastic medium, parameters such as shear wave velocity and compression wave velocity can be determined. Or in a poroelastic medium or poroelastic soft medium, parameters such as shear wave velocity, fast compression wave velocity, and slow Biot wave velocity can be determined. In a variant, a single electronic device could fulfill all the functionalities of control unit 111a and computer 111b.

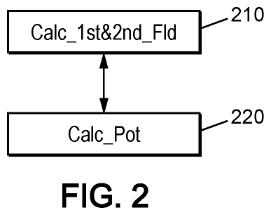

FIG. 2 describes a flow chart of the method according to the present disclosure.

In reference to FIG. 2, the method for separating a displacement vector field \vec{U} resulting from the displacement of a mechanical wave into its shear component and its compression component may comprise calculating 210 a first vector field and a second vector field from said displacement vector field \vec{U} by using the Helmholtz theorem, and the first vector field may be a function of a potential.

Indeed, by assuming that the displacement vector field \vec{U} is known in all locations within a volume of interest, the displacement vector field \vec{U}, in this volume, may be written \vec{U}(x, y, z, t), where x, y and z may represent the three axes in space, and t represents time. Typically, in elastography, the volume of interest may be an organ under examination and its neighborhood for instance.

All three components (x, y, z) of the displacement vector field are known and may be comprised in raw signal data (e.g. raw propagation images), for example, obtained with an MRI scanner equipped with a magnetic resonance elastography software according to the 3D imaging system of FIG. 1 previously presented for instance.

The displacement vector field may comprise a first vector field and a second vector field. For instance, the first vector field may correspond to a shear field relative to a shear component (i.e. a shear wave) of a mechanical wave propagating in a medium (for instance viscoelastic medium or poroelastic medium) and the second vector field may correspond to a compression field relative to a compression component (i.e. compression wave) of a mechanical wave propagating in a medium (for instance viscoelastic medium or poroelastic medium or poroelastic soft medium), or vice versa.

The decomposition allowing to obtain the first vector field and the second vector field may be performed by using the Helmholtz theorem. The Helmholtz theorem states that any smooth and rapidly decaying vector field \vec{U} may be decomposed into the sum of an irrotational (curl-free) vector field and a solenoidal (divergence-free) vector field according to the formula:

$$\vec{U} = - ∇ Φ + ∇ \times \vec{A} \qquad ( 1 )$$

In the field of mechanics, if a vector field \vec{U} is relative to a displacement field, then the curl-free component \vec{U_{P}}=−∇Φ may be relative to the compression field, i.e. the displacement field relative to the propagation of the compression wave. The divergence-free component \vec{U_{S}}=∇×\vec{A} may be relative to the shear field, i.e. the displacement field relative to the propagation of the shear wave. P is a scalar potential associated to the compression component and \vec{A} is a vector potential associated to the shear component.

After obtaining a vector field \vec{U} relative to a displacement field, the method may comprise an estimation of the first vector field based on the calculation of a potential 220.

When the potential is a vector potential, the first vector field may be the shear field \vec{U_{S}}=∇×\vec{A} and the second vector field may be the compression field \vec{U_{P}}=−∇Φ. In such embodiment, the method may include calculating the potential vector \vec{A}, then the first vector field \vec{U_{S}}, and finally calculating the second vector field \vec{U_{P}}. The method may be carried out as follows.

The vector potential \vec{A} may be calculated by solving the discrete Poisson equation (2), using any known technique, where \vec{A} is the vector potential to be determined and associated to the shear wave (or component), and the term ∇×\vec{U} at the right-hand side is known and corresponds to the curl of the displacement vector field comprised in the acquired raw signal data:

$${∇}^{2} \vec{A} = - ∇ \times \vec{U} \qquad ( 2 )$$

The demonstration of equation (2), i.e. the first Poisson Equation, may be obtained as follow:

Taking the curl of equation (1) yields:

$$∇ \times \vec{U} = - ∇ \times ( ∇ Φ ) + ∇ \times ∇ \times \vec{A} \qquad ( 3 )$$

$$∇ \times \vec{U} = ∇ \times \vec{{U}_{P}} + ∇ \times \vec{{U}_{S}} \qquad ( 4 )$$

The curl of the compressional field ∇×\vec{U_{P}} is zero because the curl of gradient is null, therefore:

$$∇ \times \vec{U} = ∇ \times \vec{{U}_{S}} = ∇ \times ∇ \times \vec{A} \qquad ( 5 )$$

Using vector identity for the curl of curl, and using the fact that \vec{A} is also divergence-free may lead to:

$$∇ \times ∇ \times \vec{A} = ∇ ( ∇ \cdot \vec{A} ) - {∇}^{2} \vec{A} = - {∇}^{2} \vec{A} \qquad ( 6 )$$

Therefore, combining equations (5) and (6) yields the well-known Poisson equation, where \vec{A} is the vector potential to be determined and which is associated to the shear wave (or component), and the term ∇×\vec{U} on the right-hand side is known and corresponds to the displacement vector field comprised in the acquired raw signal data:

$${∇}^{2} \vec{A} = - ∇ \times \vec{U}$$

In one or several embodiments, equation (2) may be solved by discretizing the Laplace operator, for instance according to the document “Numerical methods for engineers and scientists”, chapter 9.8 “Finite difference solution of the Poisson equation”, written by Joe D. Hoffman, published by Taylor & Francis Inc. in 2001, and then solving the following system for each component (x, y, z) of the vector potential \vec{A}:

$$Lx = b \qquad ( 2 )$$

In the equation (7), L is the sparse Laplace matrix, x is the vector unknown (\vec{A} reshaped as a vector), and b is a vector filled with the right-hand side of equation (2). All three components of vector \vec{A} are independent, hence the vector Poisson equation (2) may be solved as 3 independent scalar Poisson equations.

Then, once the vector potential \vec{A} is calculated (or estimated) according to above, the shear field \vec{U_{S}} (i.e. the first vector field) may be calculated (or estimated) according to the formula \vec{U_{S}}=∇×\vec{A} of the equation (1).

In one or several embodiments, the solving of the Poisson's Equation (2), i.e. the first Poisson Equation, may be rather performed in the frequency domain as presented below.

According to the following the notation where:
k²=kₓ²+k_{y}²+k_{z}² is the wave number,
U=(Ux, Uy, Uz) is the Fourier Transform of the displacement field,
A=(Ax, Ay, Az) is the Fourier Transform of the vector potential,
Us=(Usx, Usy, Usz) is the Fourier Transform of the shear component,
FT denotes the Fourier Transform, and FT⁻ denotes the inverse Fourier Transform.

In one or several embodiments, rather than the Fourier Transform or in combination, it may be used the Discrete cosine Transform (DCT) or/and the Discrete sine Transform (DST) or/and Laplace Transform.

Solving for \vec{A}, i.e. solving for the vector potential, may be performed in the frequency domain according to:

$$\vec{A} = {FT}^{- 1} ( - \frac{1}{{k}^{2}} ( \begin{matrix}{k}_{y} FT ( {U}_{z} ) - {k}_{z} FT ( {U}_{y} ) \\ {k}_{z} FT ( {U}_{x} ) - {k}_{x} FT ( {U}_{z} ) \\ {k}_{x} FT ( {U}_{y} ) - {k}_{y} FT ( {U}_{x} )\end{matrix} ) ) \qquad ( 7 )$$

Then, solving for \vec{U_{S}} (i.e. first vector field) may be performed according to:

$$\vec{{U}_{S}} = ∇ \times \vec{A}$$

Alternatively, one may solve directly for \vec{U_{S}} without calculating the potential \vec{A}, according to:

$$\vec{{U}_{S}} = {FT}^{- 1} ( - \frac{1}{{k}^{2}} ( \begin{matrix}- ( {k}_{y}^{2} + {k}_{z}^{2} ) & {k}_{x} {k}_{y} & {k}_{x} {k}_{z} \\ {k}_{y} {k}_{x} & - ( {k}_{z}^{2} + {k}_{x}^{2} ) & {k}_{y} {k}_{z} \\ {k}_{z} {k}_{x} & {k}_{z} {k}_{y} & - ( {k}_{x}^{2} + {k}_{y}^{2} )\end{matrix} ) ( \begin{matrix}FT ( {U}_{x} ) \\ FT ( {U}_{y} ) \\ FT ( {U}_{z} )\end{matrix} ) ) \qquad ( 8 )$$

The demonstration of equation (7) may be as follows. Starting from the Poisson equation (2):

$$\vec{A} = - {Δ}^{- 1} ( ∇ \times \vec{U} ) \\ FT ( \vec{A} ) = \frac{- FT ( ∇ \times \vec{U} )}{{k}^{2}} \\ FT ( \vec{A} ) = - \frac{1}{{k}^{2}} ( \begin{matrix}{k}_{y} FT ( {U}_{z} ) - {k}_{z} FT ( {U}_{y} ) \\ {k}_{z} FT ( {U}_{x} ) - {k}_{x} FT ( {U}_{z} ) \\ {k}_{x} FT ( {U}_{y} ) - {k}_{y} FT ( {U}_{x} )\end{matrix} )$$

The demonstration of equation (8) may be as follows:

$$\vec{{U}_{S}} = ∇ \times \vec{A} \\ FT ( \vec{{U}_{S}} ) = ( \begin{matrix}{k}_{y} FT ( {A}_{z} ) - {k}_{z} FT ( {A}_{y} ) \\ {k}_{z} FT ( {A}_{x} ) - {k}_{x} FT ( {A}_{z} ) \\ {k}_{x} FT ( {A}_{y} ) - {k}_{y} FT ( {A}_{x} )\end{matrix} ) \\ FT ( \vec{{U}_{S}} ) = - \frac{1}{{k}^{2}} ( \begin{matrix}{k}_{y} [ {k}_{x} FT ( {U}_{y} ) - {k}_{y} FT ( {U}_{x} ) ] - {k}_{z} [ {k}_{z} FT ( {U}_{x} ) - {k}_{x} FT ( {U}_{z} ) ] \\ {k}_{z} [ {k}_{y} FT ( {U}_{z} ) - {k}_{z} FT ( {U}_{y} ) ] - {k}_{x} [ {k}_{x} FT ( {U}_{y} ) - {k}_{y} FT ( {U}_{x} ) ] \\ {k}_{x} [ {k}_{z} FT ( {U}_{x} ) - {k}_{x} FT ( {U}_{z} ) ] - {k}_{y} [ {k}_{y} FT ( {U}_{z} ) - {k}_{z} FT ( {U}_{y} ) ]\end{matrix} ) \\ FT ( \vec{{U}_{S}} ) = - \frac{1}{{k}^{2}} ( \begin{matrix}- ( {k}_{y}^{2} + {k}_{z}^{2} ) FT ( {U}_{x} ) + {k}_{x} {k}_{y} FT ( {U}_{y} ) + {k}_{x} {k}_{z} FT ( {U}_{z} ) \\ {k}_{y} {k}_{x} FT ( {U}_{x} ) - ( {k}_{z}^{2} + {k}_{x}^{2} ) FT ( {U}_{y} ) + {k}_{y} {k}_{z} FT ( {U}_{z} ) \\ {k}_{x} {k}_{z} FT ( {U}_{x} ) + {k}_{y} {k}_{z} FT ( {U}_{y} ) - ( {k}_{x}^{2} + {k}_{y}^{2} ) FT ( {U}_{z} )\end{matrix} ) \\ FT ( \vec{{U}_{S}} ) = - \frac{1}{{k}^{2}} ( \begin{matrix}- ( {k}_{y}^{2} + {k}_{z}^{2} ) & {k}_{x} {k}_{y} & {k}_{x} {k}_{z} \\ {k}_{y} {k}_{x} & - ( {k}_{z}^{2} + {k}_{x}^{2} ) & {k}_{y} {k}_{z} \\ {k}_{z} {k}_{x} & {k}_{z} {k}_{y} & - ( {k}_{x}^{2} + {k}_{y}^{2} )\end{matrix} ) ( \begin{matrix}FT ( {U}_{x} ) \\ FT ( {U}_{y} ) \\ FT ( {U}_{z} )\end{matrix} )$$

It is interesting to note that both the Laplacian operator and the curl operations (∇×\vec{U} and ∇×\vec{A}) may be performed in the frequency domain, resulting in a simple and extremely fast solution represented by the above equation (8).

According to one or several alternatives, the calculation (or estimation) of the vector potential \vec{A} may be rather obtained by using an integral solution of the Helmholtz theorem of the equation (1) rather than using the Poisson Equation as presented above. In such case, it assumes that the displacement vector field \vec{U}(\vec{r}, t) is known everywhere inside a volume V enclosed by a closed surface S. In elastography, this surface may be the boundaries of the field of view (the region that is being investigated, typically a parallelepiped), or it may follow the boundaries of the organ of interest such liver, for example. The volume V and the surface S may also be chosen to be only a portion of particular interest within the organ of interest.

Thus, as described previously the Helmholtz theorem may be used to decompose any displacement vector field into the sum of an irrotational (curl-free) field and a solenoidal (divergence-free) field, and corresponding respectively to a compression field and a shear field, respectively. As a consequence, any displacement vector field \vec{U}(\vec{r}, t) may be written:

$$\vec{U} ( \vec{r} , t ) = {\vec{U}}_{P} ( \vec{r} , t ) + {\vec{U}}_{S} ( \vec{r} , t )$$

Where \vec{r} is the position in space, and t denotes time. According to the document “A rigorous and completed statement Helmholtz theorem” by Y. F. Gui W. B. Dou, published in year 2007 in Progress in Electromagnetics Research (PIER), volume 69, pages 287-304, the integral solution may be given by:

$$\vec{A} ( \vec{r} , t ) = {\int }_{V} \frac{∇ \times \vec{U} ( \vec{{r}^{′}} , t )}{4 π R ( \vec{r} , \vec{{r}^{′}} )} {dV}^{′} + {∮}_{S} \frac{\vec{U} ( \vec{{r}^{′}} , t ) \times \vec{n} ( \vec{{r}^{′}} , t )}{4 π R ( \vec{r} , \vec{{r}^{′}} )} {dS}^{′} \qquad ( 9 )$$

where a symbol accented with an arrow denotes a vector,
\vec{A} is the vector potential for the shear component,
\vec{r} and t are the position and time, respectively, at which the vector potential \vec{A} is being calculated,
\vec{r′} is the variable of integration and represents a moving position (it moves within V in the first integral, and over S in the second integral),
R(\vec{r}, \vec{r′})=∥\vec{r′}−\vec{r′}∥ is the distance between points \vec{r} and \vec{r′},
\vec{n}(\vec{r′}) is a unit vector at position \vec{r′}, normal to surface S, pointing outward from volume V,
\vec{curl}(\vec{u})=∇×\vec{u}=\vec{rot}(\vec{u}) is the curl operator,
\vec{u}×\vec{n}=\vec{u}∧\vec{n} denotes the cross product of vectors \vec{u} and \vec{n}.

Furthermore, in the special case where volume V is infinite, and where the displacement vector field \vec{U}(\vec{r}, t) and its partial derivatives decay rapidly toward zero at infinity, then the equation for the vector potential simplifies and becomes:

$$\vec{A} ( \vec{r} , t ) = {\int }_{V} \frac{∇ \times \vec{U} ( \vec{{r}^{′}} , t )}{4 π R ( \vec{r} , \vec{{r}^{′}} )} {dV}^{′} \qquad ( 10 )$$

Then, as presented previously, once the vector potential \vec{A} is calculated (or estimated) 220, the shear field \vec{U_{S}} (i.e. the first vector field) may be estimated (or calculated) according to the formula \vec{U_{S}}=∇×\vec{A} of the equation (1).

Then, once the first vector field is calculated (or estimated) the second vector field may be calculated from the calculated first vector field. Indeed, based on above, when the potential is a vector potential, the second vector field, i.e. the compression field \vec{U_{P}}relative to the compression wave, may be estimated (or determined or calculated) from the calculation of the shear field \vec{U_{S}} (i.e. the estimated first vector field) obtained according to one of the previous methods and using the formula \vec{U_{P}}=\vec{U}−\vec{U_{S}}.

In the method and the different approach presented previously, the compression field \vec{U_{P}} to estimate, i.e. the second vector field, may be calculated by using the estimation of the shear field \vec{U_{S}}, i.e. the calculated first vector field. However, the estimation of the compression field \vec{U_{P}}, i.e. the second vector field, may also be determined directly. Indeed, when the potential is a scalar potential, the first vector field may be the compression field \vec{U_{P}}=−∇Φ and the second vector field may be the shear field \vec{U_{S}}=∇×\vec{A}. In such embodiment, the method may rely on determining (or estimating or calculating) the scalar potential Φ, then the first vector field \vec{U_{P}}, and finally determine (or estimate or calculate) the second vector field \vec{U_{S}}. The method may be carried out as follows.

The scalar potential Φ may be calculated by solving the discrete Poisson equation (11), in a similar way to what has been already used in the present disclosure above (when the potential was a vector potential). In such embodiment, Φ is the scalar potential to be determined and associated with compression, and ∇·\vec{U} is known since \vec{U} is the displacement vector field obtained from the raw signa data (e.g. raw propagation image).

∇²Φ=−∇·\vec{U} (11)

Demonstration of equation (11) may be as follows. Taking the divergence of equation (1) yields:

\vec{U}=−∇Φ+∇×\vec{A}

∇·\vec{U}=div \vec{U}=div(−grad Φ+rot \vec{A})

=div(rot A)−div grad(Φ)

=−∇²Φ

∇²Φ=−∇·\vec{U}

The solving of the discrete Poisson equation allows to obtain an estimation 220 of the scalar potential Φ which may be then used to calculate (or estimate) the compression field \vec{U_{P}} (i.e. the first vector field) according to the following formula of the equation (1):

\vec{U_{P}}=−∇Φ

In one or several embodiments, solving of the Poisson Equation (11), i.e. the second Poisson Equation, may be rather performed in the frequency domain. Indeed, the compression vector field \vec{U_{P}} may be determined in the frequency domain, in a similar way to what has been described previously (when the potential is a vector potential), and therefore may be obtain as follows.

According to the following the notation where:

$${k}^{2} = {k}_{x}^{2} + {k}_{y}^{2} + {k}_{z}^{2} \\ U = ( Ux , Uy , Uz )$$

FT denotes the Fourier Transform.

Solving for Φ, i.e. solving for the scalar potential, may be performed in the frequency domain according to:

$$Φ = {FT}^{- 1} ( - \frac{1}{{k}^{2}} ( {k}_{x} FT ( {U}_{x} ) + {k}_{y} FT ( {U}_{y} ) + {k}_{z} FT ( {U}_{z} ) ) ) \qquad ( 12 )$$

Then, solving for \vec{U_{P}} (i.e. second vector field) may be performed according to:

$$\vec{{U}_{P}} = - ∇ Φ$$

Alternatively, solving for \vec{U_{P}} (i.e. first vector field) may be performed directly, without calculating the scalar potential, according to:

$$\vec{{U}_{P}} = {FT}^{- 1} ( \frac{1}{{k}^{2}} ( \begin{matrix}{k}_{x}^{2} & {k}_{x} {k}_{y} & {k}_{x} {k}_{z} \\ {k}_{y} {k}_{x} & {k}_{y}^{2} & {k}_{y} {k}_{z} \\ {k}_{z} {k}_{x} & {k}_{z} {k}_{y} & {k}_{z}^{2}\end{matrix} ) ( \begin{matrix}FT ( {U}_{x} ) \\ FT ( {U}_{y} ) \\ FT ( {U}_{z} )\end{matrix} ) ) \qquad ( 13 )$$

Demonstration for equation (12) may be as follows:

$$Φ = - {Δ}^{- 1} ( ∇ \cdot \vec{U} ) \\ FT ( Φ ) = \frac{- FT ( ∇ \cdot \vec{U} )}{{k}^{2}} \\ FT ( Φ ) = - \frac{1}{{k}^{2}} ( {k}_{x} FT ( {U}_{x} ) + {k}_{y} FT ( {U}_{y} ) + {k}_{z} FT ( {U}_{z} ) )$$

Demonstration for equation (13) may be as follows:

$$\vec{{U}_{P}} = - ∇ Φ \\ FT ( \vec{{U}_{P}} ) = - ( \begin{matrix}{k}_{x} FT ( Φ ) \\ {k}_{y} FT ( Φ ) \\ {k}_{z} FT ( Φ )\end{matrix} ) \\ FT ( \vec{{U}_{P}} ) = \frac{1}{{k}^{2}} ( \begin{matrix}{k}_{x}^{2} FT ( {U}_{x} ) + {k}_{x} {k}_{y} FT ( {U}_{y} ) + {k}_{x} {k}_{z} FT ( {U}_{z} ) \\ {k}_{x} {k}_{y} FT ( {U}_{x} ) + {k}_{y}^{2} FT ( {U}_{y} ) + {k}_{y} {k}_{z} FT ( {U}_{z} ) \\ {k}_{x} {k}_{z} FT ( {U}_{x} ) + {k}_{y} {k}_{z} FT ( {U}_{y} ) + {k}_{z}^{2} FT ( {U}_{z} )\end{matrix} ) \\ FT ( \vec{{U}_{P}} ) = \frac{1}{{k}^{2}} ( \begin{matrix}{k}_{x}^{2} & {k}_{x} {k}_{y} & {k}_{x} {k}_{z} \\ {k}_{y} {k}_{x} & {k}_{y}^{2} & {k}_{y} {k}_{z} \\ {k}_{z} {k}_{x} & {k}_{z} {k}_{y} & {k}_{z}^{2}\end{matrix} ) ( \begin{matrix}FT ( {U}_{x} ) \\ FT ( {U}_{y} ) \\ FT ( {U}_{z} )\end{matrix} )$$

According to one or several alternatives, the calculation of the scalar potential P may be rather obtained by using an integral solution of the Helmholtz theorem of the equation (1) rather than using the Poisson Equation as presented above. By using a similar approach that the calculation of the vector potential A using integral solution of the Helmholtz theorem presented above, the integral solution for the calculation of the scalar potential P may be given by:

$$ϕ ( \vec{r} , t ) = {\int }_{V} \frac{∇ \cdot \vec{U} ( \vec{{r}^{′}} , t )}{4 π R ( \vec{r} , \vec{{r}^{′}} )} {dV}^{′} - {∮}_{S} \frac{\vec{U} ( \vec{{r}^{′}} , t ) \cdot \vec{n} ( \vec{{r}^{′}} , t )}{4 π R ( \vec{r} , \vec{{r}^{′}} )} {dS}^{′} \qquad ( 14 )$$

where a symbol accented with an arrow denotes a vector,
ϕ is the scalar potential for the compression component,
\vec{r} and t are the position and time, respectively, at which the scalar potential ϕ is being calculated,
\vec{r′} is the variable of integration and represents a moving position (it moves within V in the first integral, and over S in the second integral),
R(\vec{r}, \vec{r′})=∥\vec{r}−\vec{r′}∥ is the distance between points \vec{r} and \vec{r′},
\vec{n}(\vec{r′}) is a unit vector at position \vec{r′}, normal to surface S, pointing outward from volume V,
div(\vec{u})=∇·\vec{u}=divergence(\vec{u}) is the divergence operator,
\vec{u}·\vec{n} denotes the dot (or scalar) product of vectors \vec{u} and \vec{n}.

Furthermore, as for the calculation of the vector potential \vec{A} using integral solution of the Helmholtz theorem, in the special case where volume V is infinite, and where the displacement vector field \vec{U}(\vec{r}, t) and its partial derivatives decay rapidly toward zero at infinity, then the equation for the scalar potential simplifies and becomes:

$$ϕ ( \vec{r} , t ) = {\int }_{V} \frac{∇ \cdot \vec{U} ( \vec{{r}^{′}} , t )}{4 π R ( \vec{r} , \vec{{r}^{′}} )} {dV}^{′} \qquad ( 15 )$$

Then, as presented previously, once the scalar potential is estimated (or calculated) 220, the compression field \vec{U_{P}} (i.e. the first vector field) may be estimated (or calculated) according to the formula \vec{U_{P}}=−∇Φ of the equation (1).

Then, once the first vector field is calculated (or estimated) the second vector field may be calculated from the calculated first vector field. Indeed, based on above, when the the potential is a scalar potential, once the first vector field is calculated (or estimated), the second vector field, i.e. the shear field \vec{U_{S}} relative to the shear wave, may be obtained from the estimation of the compression field \vec{U_{P}} (i.e. the calculated first vector field) according to \vec{U_{S}}=\vec{U}−\vec{U_{P}}.

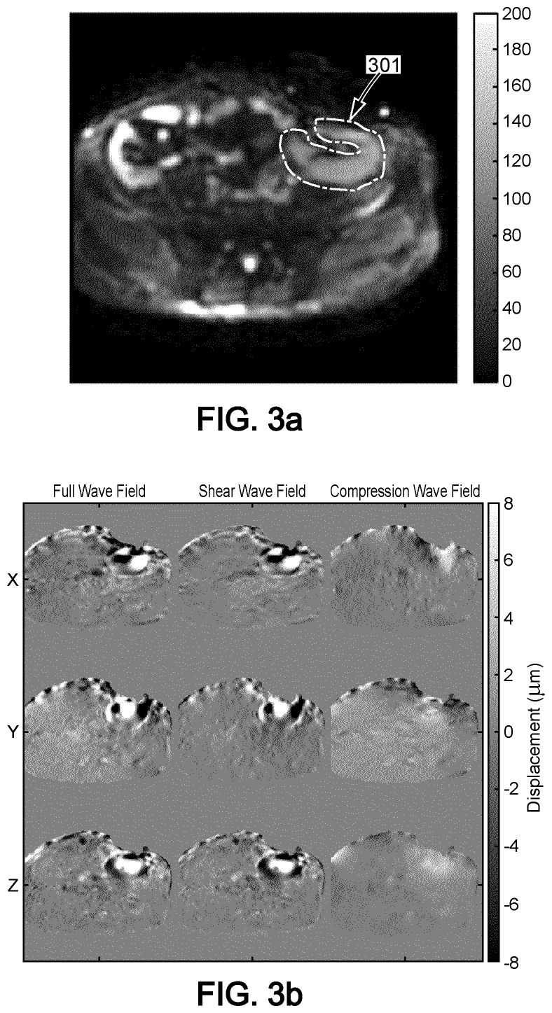

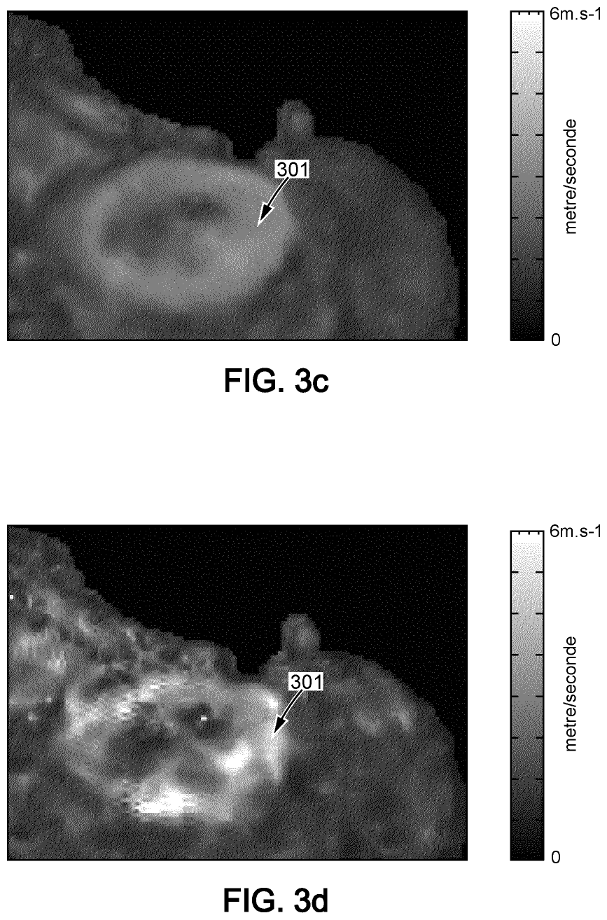

FIG. 3a illustrates an image of a medium obtained by using MRE technique.

FIG. 3b show images of shear component and compression component of a mechanical wave propagating in the medium of the FIG. 3a.

FIGS. 3c and 3d illustrate such shear wave velocity image and compression wave velocity image obtained respectively from images of shear component and of compression component of a mechanical wave propagating in a medium such wave images of FIG. 3b.

In reference to FIG. 3a, the medium of the image may be an abdomen of a patient who has received a kidney transplant. The image of the medium may have been obtained by using an 3D imaging system such as the one presented in FIG. 1. The kidney transplant is framed by the white dashed line and the numerical reference 301 on the FIG. 3a.

The kidney may be compared to a poroelastic medium leading to the generation of a fast compression wave, a slow compression waves called wave-P and a shear wave when transmitting a mechanical wave in such poroelastic medium (or poroelastic soft medium). While the fast compression wave may be ignored with conventional MRE technique, the slow compression wave (Biot slow wave) and the shear wave may have similar wave lengths in the poroelastic medium. Thus, it may be very difficult to image the respective component of each wave, and therefore difficult to determine the elastic properties of the poroelastic medium from the shear component (relative to the shear wave) or/and the compression component (relative to the slow compression wave P).

Thanks to the separation method such as the one of the present disclosure, elastography images of kidney transplant of a patient may be obtained with a better quality and for each component of the mechanical wave propagating in the poroelastic medium and with a better precision than the conventional method of filtering.

Thus, FIG. 3b shows the X, Y, and Z components of the total field (displacement field), the estimated shear field (shear component) and the estimated compressional field for an instant t retrieved (or obtained) according to the filtering method of the present disclosure. The images were acquired in the axial plane, and the field of view encompass the entire abdomen of the patient. The vibration was induced by a pneumatic vibrator located on the abdomen of the patient, directly in front of the kidney transplant. The intensity is proportional to the displacement and the units are in micrometers. X corresponds to the horizontal axis (left-right in the patient coordinate system), Y to the vertical axis (antero-posterior), and Z to the direction that passes through the imaging plane (head-feet).

From these images and by using any known inversion method (such 3D LFE for local frequency estimation for instance), tissue properties such as shear wave velocities and slow compression wave velocities can be determined independently, from the shear wave field and from the compression field, respectively, and may be used to build images of the velocity for each component such presented in FIGS. 3c and 3d.

In this example of the FIGS. 3c and 3d, such wave velocity (or speed) images are obtained by using inversion method 3D LFE on the wave images (FIG. 3b) acquired using conventional magnetic resonance elastography (MRE).

Elastic properties, such Young or/and shear modulus, may also be retrieved with a better precision, thanks to the filtering method of the present disclosure, and then allows to determine the state of the kidney transplant.

Furthermore, thanks to the separation method of the present disclosure, the information relative to each component (shear and compression) as well as the images of the velocity for each component present a higher degree of purity than the prior art. For instance, the velocity of the shear component and the shear wave velocity image(s) are not polluted (or impacted) by the information relative to the compression wave (or compression field or compression component). Likewise, the images relative to the compression field such the compression wave velocity image(s) only contain information relative to the compression waves such first fast compression wave and second slow compression wave-P—also called a PII wave or longitudinal wave of the secondary kind or Biot wave.

The first fast compression wave and the second slow compression wave-P also called Biot wave may be retrieved as follows for instance.

As described previously, in a poroelastic medium or soft poroelastic medium, the compression field may be composed of two waves: a primary (P1-) wave, and a secondary (P2-) wave. The latter is often called the Biot wave. The two P waves are superimposed, so that separating the P1-wave from the P2-wave is not straightforward.

In the specific case of biological tissues, such as liver, kidney, brain, prostate, etc., the wave speed of the P1 wave is typically on the order of 1400-1600 m/s, whereas the wave speed of the P2 wave is typically in the range 1-20 m/s. There is therefore a 100:1 ratio between these two wave speeds. This 100:1 ratio in wave speed corresponds to a 10⁴:1 ratio in elastic modulus of the P1 and P2 waves. As a consequence, div(P1)<<div(P2) by a factor of 10⁴, and div(P)=div(P1+P2)=div(P1)+div(P2)≈div(P2) because div(P1) can be neglected.

Based on this observation, and as described previously, the compression field may be estimated in two different ways, a first estimation Pa of the compression field using \vec{U_{P}}=\vec{U}−\vec{U_{S}}, and a second estimation Pb of the compression field by solving the scalar Poisson equation ∇²Φ=−∇·\vec{U} in order to then calculate \vec{U_{P}}=−∇Φ.

The first estimation Pa of the compression field may correspond to all displacements that are not caused by shear. Therefore, Pa=P1+P2. In other words, Pa contains both the P1 and the P2 waves.

The second estimation Pb of the compression field starts by estimating div(U), then by solving the corresponding scalar equation as described previously. In the specific cases of soft biological tissues (e.g. poroelastic soft medium), div(U)=div(S+P)=div(S)+div(P)≈div(P2), with S the shear component and P the compression component, and therefore the compression field Pb that gets reconstructed is mainly the P2-wave. The P1 wave is not reconstructed because its divergence is too small.

From this observation, the second estimation Pb of the compression field (described in the present disclosure) by solving the scalar Poisson equation ∇²Φ=−∇·\vec{U} in order to then calculate \vec{U_{P}}=−∇Φ may correspond to the P2-wave (P2=Pb), also called the Biot wave. And the P1 wave may be estimated from the following equation P1=Pa−Pb.

Thus, thanks to the separation method, it may be possible to retrieve each compression wave of a compression component of a mechanical wave propagating in a medium as poroelastic soft medium such biological tissues, such as liver, kidney, brain, prostate, etc., in particular to retrieve the slow wave (P2_wave or slow compression wave) called Biot wave allowing to then determine elastic properties of the medium such stiffness, viscosity, permeability of the medium which may be diagnostic markers of the studied medium as organ such as liver, kidney, brain, prostate, etc.

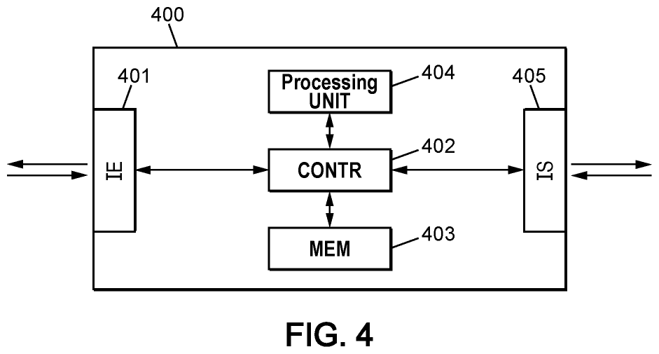

FIG. 4 illustrates an exemplary architecture of a device configured for the implementation of embodiments of the proposed scheme.

Depending on the embodiment, the architecture proposed below may be used for the system control, the control unit of the computer, of FIG. 1.

With reference to FIG. 4, the device 400 may comprise a controller 402, operatively coupled with an input interface 401, an output interface 405 and a memory 403, which may be configured to control a processing unit 404 for separating a displacement vector field comprised in a raw signal data acquired from an 3D imaging system according to the present disclosure.

The input interface 401 may be configured to receive as input at least one raw signal data comprising a displacement vector field obtained from an 3D imaging system such imaging system presented at FIG. 1. The input interface 901 may also be configured to receive information data from the mechanical wave generator,

The controller 402 may be configured to control the processing unit 404 for the implementation of one or more embodiments of the proposed method.

The processing unit 404 may be configured to perform a separation of a displacement vector field comprised in raw signal data provided by a 3D imaging system, the displacement vector field being relative to a mechanical wave propagating in a medium, the mechanical wave having a shear component and a compression component.

The device 400 may be configured to implement one or more embodiments of the proposed method for separating a displacement vector field \vec{U} resulting from the displacement of a mechanical wave into its shear component and its compression component. In particular, the device 400 may be configured for:
calculating a first vector field and a second vector field from said displacement vector field \vec{U} by using the Helmholtz theorem, and said first vector field being function of a potential, and wherein the first vector field is calculated based on the potential, and wherein the second vector field is calculated from the first vector field.

The device 400 may be a computer, a control system, a control unit (such as, for example, presented in FIG. 1), a computer network, an electronic component, or another device comprising a processor operatively coupled with a memory, as well as, depending on the embodiment, a storage unit, and other associated hardware elements such as a network interface and a media drive for reading and writing to removable storage media (not shown in the figure). Depending on the embodiment, the memory, the data storage unit or the removable storage medium contains instructions which, when executed by the controller 402, cause this controller 402 to perform or control the interface parts of input 401, the memory 403, the processing unit 404, and the output interface 405, separate a displacement vector field and/or data processing of the examples of implementation of the proposed method described herein. The controller 402 may be a component implementing a processor or a calculation unit for separating a displacement vector field according to the proposed method and the control of units 401, 402, 403, 404, 405, of device 400.

The device 400 may be implemented in software, as described above, or in hardware, such as an application specific integrated circuit (ASIC), or in the form of a combination of hardware and software, such as for example a software program intended to be loaded and executed on a component of FPGA (Field Programmable Gate Array) type.

1-25. (canceled)

26. A method for separating a displacement vector field U resulting from the displacement of a mechanical wave in a medium into its shear component and its compression component, said displacement of the mechanical wave being measured by an imaging device configured for acquiring the displacement vector field relative to the mechanical waves propagating in the medium, wherein the method comprises:
calculating a first vector field and a second vector field from said displacement vector field \vec{U} by using the Helmholtz theorem, and said first vector field being function of a potential,
and wherein the first vector field is calculated based on the potential, and wherein the second vector field is calculated from the first vector field.

27. Method according to claim 26, wherein the potential is a vector potential or a scalar potential.

28. Method according to claim 27, wherein when the potential is a vector potential, the method further comprising:
calculating an output resulting from the application of a curl operator on a decomposition's formula \vec{U}=−∇Φ+∇×\vec{A} obtained from the Helmholtz theorem, where said ∇×\vec{A}=\vec{U_{S}} is the first vector field relative to the shear component and −∇Φ=\vec{U_{P}} is the second vector field relative to the compression component, the said output corresponding to a first Poisson Equation according to the formula ∇₂\vec{A}=−∇×\vec{U},
and wherein the vector potential \vec{A} is calculated by solving numerically said first Poisson Equation.

29. Method according to claim 27, wherein when the potential is a vector potential, the vector potential is calculated by using an integral solution of Helmholtz theorem according to the formula:
$$\vec{A} ( \vec{r} , t ) = {\int }_{V} \frac{∇ \times \vec{U} ( \vec{{r}^{′}} , t )}{4 π R ( \vec{r} , \vec{{r}^{′}} )} {dV}^{′} + {∮}_{S} \frac{\vec{U} ( \vec{{r}^{′}} , t ) \times \vec{n} ( \vec{{r}^{′}} , t )}{4 π R ( \vec{r} , \vec{{r}^{′}} )} {dS}^{′}$$
wherein,
\vec{A} is the vector potential,
\vec{r} and tare the position and time, respectively, at which the vector potential \vec{A} is being calculated,
\vec{r′} is the variable of integration and represents a moving position (it moves within V in the first integral, and over S in the second integral),
R(\vec{r}, \vec{r′})=∥\vec{r}−\vec{r′}∥ is the distance between points \vec{r} and \vec{r′},
\vec{n}(\vec{r′}) is a unit vector at position \vec{r′}, normal to surface S, pointing outward from volume V,
∇×\vec{U} is the curl operator,
\vec{U}×\vec{n}=\vec{U}∧\vec{n} denotes the cross product of vectors \vec{U} and \vec{n}.

30. Method according to claim 28, wherein the first vector field \vec{U_{S}} is calculated according to the formula \vec{U_{S}}=∇×\vec{A}.

31. Method according to claim 29 wherein the first vector field \vec{U_{S}} is calculated according to the formula \vec{U_{S}}=∇×\vec{A}.

32. Method according to claim 28 wherein the second vector field \vec{U_{P}} is calculated according to the formula \vec{U_{P}}=\vec{U}−\vec{U_{S}}.

33. Method according to claim 29 wherein the second vector field \vec{U_{P}} is calculated according to the formula \vec{U_{P}}=\vec{U}−\vec{U_{S}}.

34. Method according to claim 27 wherein when the potential is a scalar potential, the method further comprising:
calculating an output resulting from the application of a divergence operator on the decomposition's formula \vec{U}=−∇Φ+∇×\vec{A} obtained from the Helmholtz theorem, where said −∇Φ=\vec{U_{P}} is the first vector field relative to the compression component and said ∇×\vec{A}=\vec{U_{S}} is the second vector field relative to the shear component, the said output corresponding to a second Poisson Equation according to the formula ∇²Φ=−∇·\vec{U},
and wherein the scalar potential Φ is calculated by solving numerically said second Poisson Equation.

35. Method according to claim 27, wherein when the potential is a scalar potential, the scalar potential is calculated by using an integral solution of the Helmholtz theorem according to the formula:
$$ϕ ( \vec{r} , t ) = {\int }_{V} \frac{∇ \cdot \vec{U} ( \vec{{r}^{′}} , t )}{4 π R ( \vec{r} , {r}^{′} )} {dV}^{′} - {∮}_{S} \frac{\vec{U} ( \vec{{r}^{′}} , t ) \cdot \vec{n} ( \vec{{r}^{′}} , t )}{4 π R ( \vec{r} , \vec{{r}^{′}} )} {dS}^{′}$$
wherein,
ϕ is the scalar potential,
\vec{r} and t are the position and time, respectively, at which the scalar potential ϕ is being calculated,
\vec{r′} is the variable of integration and represents a moving position (it moves within V in the first integral, and over S in the second integral),
R(\vec{r}, \vec{r′})=∥\vec{r}−\vec{r′}∥ is the distance between points \vec{r} and \vec{r′},
\vec{n}(\vec{r′}) is a unit vector at position \vec{r′}, normal to surface S, pointing outward from volume V,
∇·\vec{U} is the divergence operator,
\vec{U}·\vec{n} denotes the dot (or scalar) product of vectors \vec{U} and \vec{n}.

36. Method according to claim 34 wherein the first vector field \vec{U_{P}} is calculated according to the formula \vec{U_{P}}=−∇Φ.

37. Method according to claim 35 wherein the first vector field \vec{U_{P}} is calculated according to the formula \vec{U_{P}}=−∇Φ.

38. Method according to claim 34 wherein the second vector field \vec{U_{S}} is calculated according to the formula \vec{U_{S}}=\vec{U}−\vec{U_{P}}.

39. Method according to claim 35 wherein the second vector field \vec{U_{S}} is calculated according to the formula \vec{U_{S}}=\vec{U}−\vec{U_{P}}.

40. Method according to claim 28 wherein solving of the first Poisson Equation or the second Poisson Equation is performed in the frequency domain.

41. Method according to claim 34 wherein solving of the first Poisson Equation or the second Poisson Equation is performed in the frequency domain.

42. Method according to claim 28 wherein the first Poisson Equation or the second Poisson Equation is a discrete Poisson equation.

43. Method according to claim 34 wherein the first Poisson Equation or the second Poisson Equation is a discrete Poisson equation.

44. Method according to claim 26, wherein the medium is any medium that allows the propagation of shear and compression waves, including a viscoelastic medium, a poroelastic medium, a poro-visco-elastic medium, a composite medium or a poro-composite medium.

45. Method according to claim 44, wherein the compression component is relative to at least one slow compression wave called Biot wave and to at least one fast compression wave.

46. Method according to claim 45, wherein when \vec{U_{P}} as first vector field is determined from \vec{U_{P}}=−∇Φ and the second Poisson Equation according to the formula ∇²=−∇·\vec{U}, \vec{U_{P}} is relative to the at least one slow compression wave called Biot wave.

47. Method according to claim 45, wherein the at least one fast compression wave is determined from the at least slow compression wave.

48. A 3D imaging system for imaging a first vector field and second vector field comprised in a displacement field relative to a mechanical wave propagating in a medium, the 3D imaging system comprising:
an imaging device configured to acquire the displacement vector field relative to the mechanical waves propagating in the medium,
a control system configured for acquiring at least one raw signal data comprising the displacement vector field, for separating the displacement vector field according to claim 1 to obtain a first vector field and a second vector field, and for generating a 3D image of the first vector field and the second vector field.

49. 3D imaging system according to claim 48 wherein the control system is further configured to use the 3D image of the first vector field to determine a length and/or a speed or/and an image of speed of the wave relative the first vector field, and configured to use the 3D image of the second vector field to determine a length and/or a speed or/and an image of speed of the wave relative the second vector field.

50. 3D imaging system according to claim 48, wherein the medium may be any medium that allows the propagation of shear and compression waves, including a viscoelastic medium, a poroelastic medium, a poro-visco-elastic medium, a composite medium or a poro-composite medium.

51. 3D imaging system according to claim 48, wherein the imaging device is a Magnetic Resonance Imaging configured to perform Magnetic Resonance Elastography.

52. Computer software comprising instructions to implement at least a part of a method according to claim 26 when the software is executed by a processor.

53. Computer-readable non-transient recording medium on which a software is registered to implement a method according to claim 26 when the software is executed by a processor.
